(19) United States
(12) Patent Application Publication
(10) Pub. No.: US 2002/0114851 A1
(43) Pub. Date: Aug. 22, 2002

(54) Therapeutic mouthwash containing alkali metal hypohalite

(76) Inventors: Jurdon Wayne Camper, Rolla, ND (US); Marilyn A. Delorme, Bellcourt, ND (US); John E. Festvog, Rolla, ND (US)

Correspondence Address: DORSEY & WHITNEY LLP, 801 GRAND, SUITE 3900, DES MOINES, IA, 50309, US

(21) Appl. No.: 09/784,216

(22) Filed: Feb. 16, 2001

(51) Int. Cl. A61K 33/14

(52) U.S. Cl. 424/665; 424/661

(57) ABSTRACT

A mouthwash or oral rinse containing a mixture of an aqueous solution and an alkali metal hypohalite (e.g., sodium hypochlorite). Optionally, the aqueous solution contains a flavor masking or neutralizing agent such as fruit juice. The user rinses the oral cavity or gargles with the mouthwash to treat gingivitis or kill odor or plaque causing bacteria, or to eliminate viruses, fungi and/ or other microorganisms in the oral cavity.

### FIELD OF THE INVENTION

The instant invention relates to a mouthwash. More specifically, the invention relates to a mouthwash or oral rinse that contains an alkali metal hypohalite (AMH), e.g., sodium hypochlorite. The AMH kills bacteria, fungi, and viruses in the mouth and throat.

### BACKGROUND ART

The most common AMH is sodium hypochlorite (NaOCl). Sodium hypochlorite is the active agent in household bleach. Due to its bactericidal properties, it is also a common ingredient in swimming pool disinfectants and water purification treatments. See Hawley's Condensed Chemical Dictionary 13th ed., by Richard R. Lewis, John Wiley & Sons, New York, N.Y., page 1019 (1997).

A number of compositions containing sodium hypochlorite have been patented. Representative patents include U.S. Pat. Nos.: 4,737,307; 4,927,641; 5,273,678; 5,472,714; and 5,427,801.

U.S. Pat. No. 4,737,307 is directed to a skin cleanser capable of removing smegma and surface bacteria fungus and viruses from the skin. The cleanser is an aqueous solution of cetylpyridinium chloride, chlorine dioxide, sodium hypochlorite, polyoxyethylene (20) sorbitan monostearate and sodium benzoate.

U.S. Pat. No. 4,927,641 is directed to a veterinary liniment. The liniment comprises dimethlysulfoxide and sodium hypochlorite. The liniment is preferably a thin liquid but may be in the form of a lotion, gel, or cream.

U.S. Pat. No. 5,273,678 is directed to a stable aqueous solution containing sodium hypochlorite, a pH regulator, and water. The solution is used as an antiseptic.

U.S. Pat. Nos. 5.472,715 and 5,427,801 are directed to an antifungal agent for the treatment of surface skin diseases such as athlete's foot, ringworm and tinea caused by dermatophytes, eczema, tinea or various fungi. The agent comprises a detergent solution of sodium hypochlorite, sodium sulfite, sodium nitrate, sodium chlorate, potassium chlorate, hydrogen peroxide, oxone water, sodium nitrite, potassium nitrite, nonionic surface active agent, and water.

Oral rinse (or mouthwash) compositions are known and used for the prevention of bad breath and for the elimination of bacteria and other microorganisms responsible for bad breath, tooth decay, plaque and gum diseases such as gingivitis and periodontitis. Conventional mouthwashes usually employ ethyl alcohol with percentages ranging from approximately 10% up to about 30% by volume. The alcohol serves as a disinfectant and as a solvent in which other additives such as astringents, fluorides, color additives, flavor oils, bactericidal actives and the like can be dissolved or dispersed.

The prior art does not appear to teach a mouth wash containing AMH. U.S. Pat. No. 6,077,502 is directed to an oral care composition containing chlorite ion. However, the composition is preferably free of hypochlorite metal salt or hypochlorite ion.

### SUMMARY OF THE INVENTION

The present invention comprises a mouthwash (or oral rinse) with which the user rinses his or her oral cavity. The mouthwash is an aqueous solution that contains AMH as the active ingredient. The mouthwash eliminates bacteria and other microorganisms and, thereby, prevents bad breath, tooth decay, plaque and gum diseases such as gingivitis and periodontitis as well as viral infections (e.g., cold sores).

### DETAILED DESCRIPTION OF THE INVENTION

The oral rinse or mouthwash of the instant invention comprises: an aqueous base and AMH. Preferably, these are the only components of the mouthwash or oral rinse. For the purposes herein, oral rinse and mouthwash are used interchangeably.

The preferred AMH is sodium hypochlorite. However, other AMHs, such as calcium hypochlorite, may also be utilized. Sodium hypochlorite is commonly available in solution form. The preferred sodium hypochlorite solution is 7.25% sodium hypochlorite by weight.

Any of a number of aqueous bases may be employed, including pure water. However, it is preferred to use some sort of flavored base, e.g., grapefruit juice, since this serves to neutralize or mask the unpleasant taste of the AMH. However, the flavorant in the aqueous base is not critical. Any of a wide variety of natural and artificial flavorants may be employed or the flavorant may be omitted all together in favor of a pure aqueous base.

The mouthwash is prepared by adding the AMH to the aqueous base. Specifically, anywhere from 0.5 to 10 milliliters (1/10 to 2 teaspoons) of 7.25%, by volume, AMH solution is added to 473.2 milliliters (one pint) of aqueous base. This corresponds to a range of AMH in the mouthwash of about 0.0076% by volume and about 0.15% by volume. However, mouthwashes containing concentrations of pure AMH ranging as high as about 6.33% by volume have proven both safe and effective.

The preferred embodiment is prepared by adding 5 ml of a 7.25% by volume sodium hypochlorite solution to one pint of grapefruit juice. This corresponds to a mouthwash containing 0.075% by volume pure sodium hypochlorite.

Preferably, the AMH is the principal active ingredient in the mouthwash. More preferably, the AMH is the only active ingredient in the mouthwash.

Other common mouthwash additives may be added. Such additives include flavorants, colorants, fluorides, and calcium fortifying agents.

In practice, the user rinses or gargles with the mouthwash at least once a day. However, the number of rinses needed varies between individuals. More severe cases of gingivitis may require more frequent rinses.

What is claimed is:

1. A mouthwash composition comprising an alkali hypohalite dissolved in an aqueous base wherein the concentration of alkali hypohalite is between about 0.0076 and about 6.33% by volume.

2. The composition of claim 1, wherein the alkali hypohalite is sodium hypochlorite.

3. The composition of claim 1, wherein the mouthwash additionally contains an odor or taste masking agent.

4. The composition of claim 3, wherein the odor or taste masking agent is a fruit juice.

5. The composition of claim 4, wherein the fruit juice is grapefruit juice.

6. The composition of claim 2, wherein the concentration of sodium hypochlorite is 0.075% by volume.

7. The composition of claim 2, wherein the sodium hypochlorite is the principal active ingredient.

8. The composition of claim 2, wherein the aqueous base is water.
